(19) United States
(12) Patent Application Publication
(10) Pub. No.: US 2023/0109649 A1
(43) Pub. Date: Apr. 6, 2023

(54) METHODS AND SYSTEMS FOR GENERATING BIOLOGICAL MOLECULES

(71) Applicant: Machine Bio Inc., Fountain Hills, AZ (US)

(72) Inventor: David MARASH, Fountain Hills, AZ (US)

(21) Appl. No.: 17/744,136

(22) Filed: May 13, 2022

Related U.S. Application Data
(63) Continuation of application No. PCT/US20/60585, filed on Nov. 13, 2020.
(60) Provisional application No. 62/935,637, filed on Nov. 15, 2019.

(51) Int. Cl. C12P 21/02

(52) U.S. Cl. CPC C12P 21/02

(57) ABSTRACT

The present disclosure provides methods and systems for generating biological molecules. The methods and systems may comprise use of a porous membrane. The present disclosure also provides methods and systems of generating porous membranes.

### CROSS-REFERENCE

This application is a continuation of International Patent Application No. PCT/US20/60585 filed on Nov. 13, 2020, claims the benefit of U.S. Provisional Patent Application No. 62/935,637, filed Nov. 15, 2019, which is entirely incorporated herein by reference for all purposes.

### BACKGROUND

The ability to produce molecules on demand can have significant implications in various fields such as pharmaceuticals and life sciences research. Manufactured biomolecules can contain impurities that can increase the cost of the preparation of the biomolecules as well as the time taken to prepare the biomolecules. In particular, the cost of preparing a protein can mostly be the cost of purifying the protein from the reaction mixture.

### SUMMARY

Recognized herein is the need for improved biological molecule synthesis approaches that may enable higher purity products with less intensive operating conditions. The present disclosure provides methods and systems for generating a biological molecule, such as a polypeptide or a protein. Methods and systems of the present disclosure may enable the formation of a biological molecule at a high purity (e.g., a purity of at least 60%, 70%, 80%, 90%, 95% or greater).

In an aspect, the present disclosure provides a cell-free biological molecule reaction system with a membrane comprising translocon and/or signal peptidase proteins. The translocon proteins can provide a selective channel that permits movement of biological molecules synthesized in a cell-free reaction solution through a membrane while not permitting movement of impurity molecules. The signal peptidase molecules can cleave signal regions from the biological molecules to release the molecules from the membrane and allow the molecules to be collected. This membrane-based system can generate biological molecules at a significantly higher purity as compared to cell-free synthesis alone and can provide new reaction engineering conditions due to the presence of two reaction zones.

In another aspect, the present disclosure provides a method for generating a biological molecule, comprising: (a) providing a chamber comprising a first portion comprising a plurality of cell-free precursors of said biological molecule, a second portion, and a membrane separating said first portion from said second portion, wherein said membrane comprises a pore; (b) using at least a subset of said plurality of cell-free precursors from said first portion to form said biological molecule; and (c) during or subsequent to (b), translocating at least a portion of said biological molecule through said pore into said second portion.

In some embodiments, said membrane comprises a lipid bilayer. In some embodiments, said lipid bilayer is a supported lipid bilayer. In some embodiments, said lipid bilayer comprises one or more translocon proteins. In some embodiments, the method further comprises (d) removing said biological molecule from said second portion of said chamber. In some embodiments, said removing comprises at most about two purification operations. In some embodiments, said removing does not comprise a purification operation. In some embodiments, said biological molecule further comprises an N-terminal translocation signal sequence. In some embodiments, subsequent to (c), said N-terminal translocation signal sequence is removed from said biological molecule. In some embodiments, said translocating occurs substantially simultaneously to said forming said biological molecule. In some embodiments, said translocating occurs subsequently to said forming said biological molecule. In some embodiments, said translocating occurs co-translationally. In some embodiments, said biological molecule is a polypeptide. In some embodiments, said polypeptide is a protein, and wherein at least a portion of said protein is formed in said first portion and folded in said second portion. In some embodiments, said pore has a cross section that is larger than a cross section of said biological molecule. In some embodiments, said chamber is a part of a flow channel. In some embodiments, said cell-free precursors do not comprise said biological molecule. In some embodiments, (c) comprises translocating an entirety of said biological molecule through sad pore and into said second portion subsequent to (b). In some embodiments, (c) is performed during (b). In some embodiments, (c) is performed subsequent to (b).

In another aspect, the present disclosure provides a system for generating a biological molecule, comprising: a chamber comprising a first portion configured to comprise a plurality of cell-free precursors of said biological molecule; a second portion; and a porous membrane separating said first portion from said second portion, wherein said porous membrane comprises a lipid bilayer, and wherein said lipid bilayer comprises one or more translocon proteins.

In some embodiments, said lipid bilayer is a supported lipid bilayer. In some embodiments, said porous membrane comprises hydrophilic polysulfone, mesoporous silica, or mesoporous alumina. In some embodiments, said hydrophilic polysulfone has a molecular weight cut off of at most about 100 kilodaltons. In some embodiments, said one or more translocon proteins comprise one or more proteins selected from the group consisting of SecYEG, SecY, SecE, SecG, Sec61p, and an injectosome. In some embodiments, said plurality of cell-free precursors do not comprise one or more cells. In some embodiments, said plurality of cell-free precursors comprises deoxyribonucleic acid (DNA). In some embodiments, said DNA encodes for said biological molecule. In some embodiments, said biological molecule is a protein, and wherein said second portion comprises conditions for optimal folding of said protein. In some embodiments, said biological molecule is a nucleic acid molecule, a protein, an antigen, a polypeptide, an enzyme, or a chemical. In some embodiments, said supported lipid bilayer comprises one or more signal peptidase proteins.

In another aspect, the present disclosure provides a method for generating a cell-free synthesis chamber, comprising: (a) providing a chamber comprising a first portion and a second portion, wherein said first portion and said second portion are separated by a porous membrane; (b) applying a solution comprising a plurality of proteoliposomes, wherein said plurality of proteoliposomes comprise a lipid bilayer and one or more translocon proteins; and (c) reacting said plurality of proteoliposomes with said porous membrane, wherein said reacting comprises dissociation of said plurality of proteoliposomes to form a lipid bilayer on said porous membrane, wherein said lipid bilayer comprises said one or more translocon proteins.

In some embodiments, said lipid bilayer is a supported lipid bilayer. In some embodiments, said solution comprises a plurality of liposomes without said one or more translocon proteins. In some embodiments, a concentration of said one or more translocon proteins is controlled by a ratio of said proteoliposomes to said plurality of liposomes. In some embodiments, said proteoliposomes are substantially homogenous in size. In some embodiments, said proteoliposomes are generated by incubation of liposomes with cell-free precursors of said translocon proteins.

In another aspect, the present disclosure provides a method for generating a polypeptide, comprising (a) using a cell-free solution comprising a deoxyribonucleic acid molecule encoding said polypeptide to generate a ribonucleic acid molecule, (b) using said ribonucleic acid molecule to generate said polypeptide, and (c) directing said polypeptide through a pore disposed in a membrane.

In some embodiments, subsequent to (c), said polypeptide is present at a purity of at least 60%. In some embodiments, (a)-(c) is performed in a time period of at most 1 day. In some embodiments, said membrane is not a part of a micelle. In some embodiments, said membrane is planer. In some embodiments, said polypeptide comprises a non-native N-terminal signal sequence. In some embodiments, said pore comprises one or more translocon proteins. In some embodiments, said membrane comprises one or more signal peptidase proteins. In some embodiments, said polypeptide is a protein.

In another aspect, the present disclosure provides a system for generating a biological molecule, comprising: a chamber comprising a first portion configured to comprise a plurality of cell-free precursors of said biological molecule; a second portion; and a porous membrane separating said first portion from said second portion, wherein said porous membrane comprises a lipid bilayer, and wherein said lipid bilayer comprises one or more signal peptidase proteins.

In some embodiments, said one or more signal peptidase proteins comprise LepB. In some embodiments, said lipid bilayer further comprises one or more translocon proteins. In some embodiments, said lipid bilayer is a supported lipid bilayer.

In another aspect, the present disclosure provides a system for generating a biological molecule, comprising: a chamber comprising a first portion configured to comprise a plurality of cell-free precursors of said biological molecule, a second portion, and a membrane separating said first portion from said second portion, wherein said membrane comprises a pore; a controller comprising one or more computer processors that are individually or collective configured to direct a method for generating said biological molecule, said method comprising: (i) using at least a subset of said plurality of cell-free precursors from said first portion to form said biological molecule; and (ii) during or subsequent to (i), translocating at least a portion of said biological molecule through said pore into said second portion.

In some embodiments, said method further comprises removing said biological molecule from said second portion of said chamber. In some embodiments, said method further comprises removing an N-terminal translocation signal sequence. In some embodiments, said translocating occurs substantially simultaneously to said forming said biological molecule. In some embodiments, said translocating occurs subsequently to said forming said biological molecule. In some embodiments, said translocating occurs co-translationally.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic of an example process for generating a biological molecule.

FIG. 2 is an example of a flowchart for a process for generating a cell-free synthesis chamber.

FIG. 3 is an example flowchart for a process for generating a polypeptide.

FIGS. 4A-4D are examples of a method for generating a flow cell chamber

FIG. 5 is an example of a supported lipid bilayer comprising proteins.

FIGS. 6A-6B are examples of a process for generating a chamber comprising a membrane and using the chamber to generate a biological molecule.

FIGS. 7A-7C are examples of a process for generating a biological molecule.

FIG. 8 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “pore,” as used herein, generally refers to a channel or conduit capable of permitting a substance to move from one location to another location. The pore may have at least one opening. In some examples, the pore has at least two openings. The pore can have a cross-section (e.g., diameter) that is on the micrometer or nanometer scale. The pore can have a cross-section that is at most 1 micrometer (um), 500 nanometers (nm), 400 nm, 300 nm, 200 nm, 100 nm, or smaller. The cross-section can be sized to be larger than a longest cross-section of a biological molecule (e.g., polypeptide or protein) to be formed. The pore can be a nanopore (e.g., a pore having a cross-section that is at most 1 um). The pore can be part of a biological molecule, such as a protein (e.g., alpha-hemolysin, a translocon protein, etc.) (e.g., a biological material comprising a pore embedded in a lipid bilayer), or part of a solid-state material, such as, for example, a dielectric (e.g., the pore may be formed within the dielectric), or a combination thereof (e.g., a biological molecule comprising a pore can be positioned over a pore in a solid-state material).

The term “polypeptide,” as used herein, generally refers to a biological molecule comprising at least two amino acids. The polypeptide can be a protein.

The term “cell-free,” as used herein, generally refers to a material that is external to a cell. A cell-free material may be released from the cell, such as, for example, upon lysis or permeabilization of a cell. The cell-free material may have been generated in an environment external to the cell (e.g., generated in a reactor, generated by external proteins of a cell, etc). The cell-free material may be provided or generated in a cell-free environment in which one or more components of the cell (e.g., intracellular components, such as, for example, enzymes, ribosomes, etc.) are present.

In an aspect, the present disclosure provides a method for generating a biological molecule. The biological molecule may be a polypeptide or a nucleic acid molecule, for example. The biological molecule may be a polypeptide (e.g., protein). The biological molecule may be a nucleic acid molecule, such as a deoxyribonucleic acid (DNA) or ribonucleic acid (RNA) molecule.

A method for generating a biological molecule may comprise providing a chamber comprising a first portion containing a plurality of cell-free precursors of the biological molecule, a second portion, and a membrane separating the first portion from the second portion. The membrane may comprise a pore. At least a subset of the plurality of cell-free precursors may be used from the first portion to form the biological molecule. The biological molecule may be translocated through the pore into the second portion.

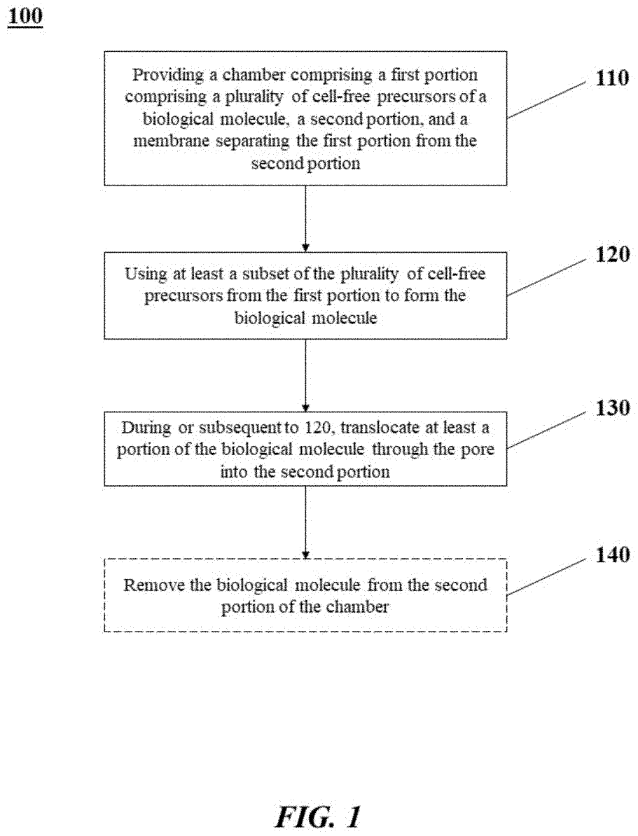

FIG. 1 is a schematic of an example process 100 for generating a biological molecule. In an operation 110, the process 100 may comprise providing a chamber comprising a first portion comprising a plurality of cell-free precursors of a biological molecule, a second portion, and a membrane separating the first portion from the second portion.

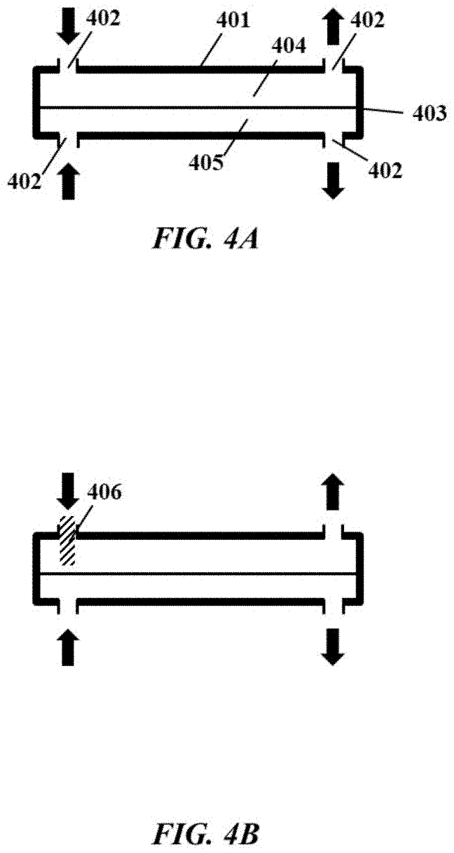

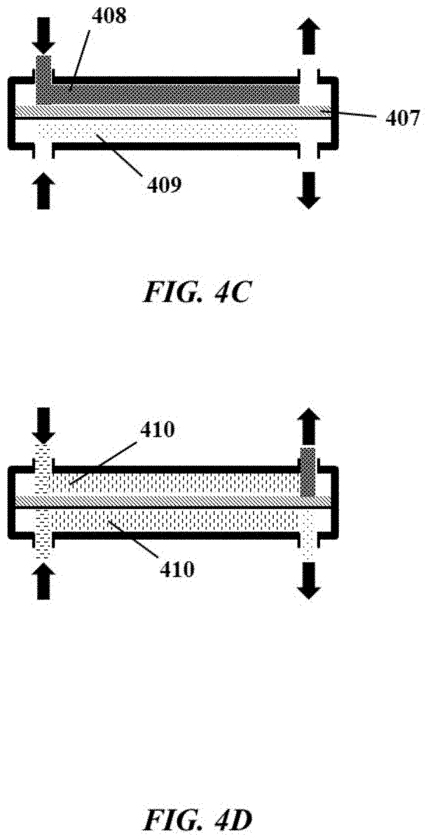

The chamber may be formed of plastic (e.g., polyethylene, polystyrene, resin, polytetrafluoroethylene, etc.), metal (e.g., aluminum, iron, copper), fiber-based materials (e.g., carbon fiber, etc.), or the like, or any combination thereof. The chamber may comprise a plurality of portions. The chamber may comprise at least about 2, 3, 4, 5, 6, 7, 8, 9, 10, or more portions. The chamber may comprise at most about 10, 9, 8, 7, 6, 5, 4, 3, or fewer portions. The chamber may be configured with environmental control apparatuses (e.g., temperature controllers, pressure controllers, etc.), monitoring apparatuses (e.g., thermocouples, pH meters, optical spectroscopy instruments, etc.), electrodes, or the like, or any combination thereof The chamber may be a part of a flow channel. For example, the chamber may be a part of a flow channel as shown in FIGS. 4A-4D. In some cases, the chamber may not comprise a porous membrane. For example, instead of the chamber being configured to contain a supported lipid bilayer, the chamber can instead be configured with an unsupported lipid bilayer. The chamber may comprise a droplet microfluidic system. For example, the chamber may be a flow chamber configured to separate individual droplets comprising cell-free precursor solutions. The chamber may comprise a plurality of wells. The plurality of wells may be configured to contain a plurality of lipid bilayers. The plurality of wells may be configured such that upon raising a fluid level in the plurality of wells, the plurality of lipid bilayers can be brought into contact to form a single lipid bilayer for use as described elsewhere herein.

A plurality of chambers may be coupled together in series or in parallel. For example, a plurality of chambers can be connected in parallel to improve the throughput of generating biological molecules. In another example, a plurality of chambers can be connected in series, where the product of a first chamber can be used as a reagent in a second chamber. In this example, the biological molecule may be post-translationally modified or incorporated into a biofunctionalized scaffold. The plurality of chambers coupled together in series may be configured such that later chambers comprise one or more analysis instruments configured to analyze the biological molecule. In this way, the plurality of chambers may be configured as a lab-on-a-chip. Subsequent chambers of the plurality of chambers may be configured to biofunctionalize the biological molecule, conjugate bioactive elements to the biological molecule, or the like, or any combination thereof. The biological molecule may be analyzed by co-expression of analysis biological molecules in the first portion of the chamber, and subsequent reaction of the biological molecule with the analysis biological molecules. For example, multiple DNA templates can be expressed at the same time in the first portion, resulting in a plurality of different proteins that can translocate to the second portion and react to form a detectable complex.

The flow channel chamber may comprise a hollow fiber reaction chamber. For example, the chamber may be a hollow fiber configured with translocon proteins within the walls of the fiber configured to remove biological molecules from the fiber. The flow channel may terminate in a dead-end chamber. The dead-end chamber may be configured to accumulate biological molecules for removal. The dead-end chamber may be configured with one or more analysis instruments as described elsewhere herein. The dead-end chamber may comprise a removable chamber. The removable chamber may be a spin plate, a spin column, a filtered chamber, or the like, or any combination thereof.

The membrane may comprise a supported lipid bilayer. The supported lipid bilayer may be supported on the membrane. For example, a supported lipid bilayer can be formed on the membrane. In this example, the supported lipid bilayer can traverse a pore in the membrane. The supported lipid bilayer may comprise one or more molecules comprising a hydrophilic head and a hydrophobic tail. Examples of molecules that may form the supported lipid bilayer include, but are not limited to, phospholipids (e.g., phosphatidylcholines, 1-palmitoyl-2-oleoyl-sn-glycero-3-phosphocholine, dipalmitoylphosphatidylcholine), substituted phospholipids (e.g., phospholipids with one or more substituent groups), fatty acids (e.g., carboxylic acids), prenols, sterols, saccharolipids, polyketides, glycerolipids, sphingolipids, other lipids, or the like, or any combination thereof The membrane may comprise a pore. The pore may be a protein within the supported lipid bilayer. For example, a transmembrane protein can be positioned above a pore in the membrane to form a pore configured to permit translocation of the biological molecule from the first portion to the second portion. The pore may have a cross section that is larger than a cross section of the biological molecule. The pore may have a cross section that is larger than a cross section of a denatured conformation of the biological molecule. For example, the pore can be large enough to permit a denatured protein to traverse the pore. The pore may have a cross section larger than a cross section of a homo-oligomeric complex or a hetero-oligomeric complex. The homo- or hetero-oligomeric complex may be a complex formed by an oligomerization of the biological molecule. For example, a polypeptide can oligomerize with other polypeptides, and the pore can have a cross section larger than the oligomer.

The supported lipid bilayer may comprise one or more proteins. The one or more proteins may be configured to translocate the biological molecule across the membrane. For example, the biological molecule can pass through a pore in the membrane formed by a protein. The one or more proteins may comprise one or more translocon proteins. For example, the one or more translocon proteins may comprise SecYEG. In another example, the one or more translocon proteins may comprise Sec61p. The one or more proteins may comprise one or more injectosomes. The one or more proteins may comprise one or more hemolysins (e.g., alpha-hemolysin). The one or more proteins may comprise one or more pore forming toxins. The one or more proteins may comprise one or more signal peptidases, signal peptide hydrolases, adenosine triphosphate sythases, enzymes configured to perform post translational modifications, other chaperones, areolysins (e.g., to perform protein sequencing), or the like, or any combination thereof. The supported lipid bilayer may comprise two or more supported lipid bilayers. The two or more supported lipid bilayers may have different proteins from one another.

The biological molecule may be an antibody, an antibody binding protein, a protein, a macromolecule, an enzyme, a nucleic acid molecule, a carbohydrate, a polypeptide, a chemical, or the like, or any combination thereof The biological molecule may be a polypeptide. The polypeptide may be at least a portion of a protein. The biological molecule may be a biologically active molecule. The biologically active molecule may be a pharmaceutical molecule. For example, a small molecule therapeutic can be generated in the first portion and purified by translocation to the second portion. In another example, a pharmacologically active antibody can be generated in the first portion and subsequently translocated into the second portion where if undergoes folding to become pharmacologically active. The chemical may be a small molecule, a pharmacologically active protein, a toxin, or the like, or any combination thereof

The biological molecule may comprise a terminal translocation signal sequence. The terminal translocation signal sequence may be an N-terminal translocation signal sequence. The terminal translocation signal sequence may be configured to enable the biological molecule to translocate through the pore. For example, the terminal translocation signal sequence can be a signal sequence for a natively translocated protein. In this example, the terminal translocation signal sequence can permit movement of the biological molecule through the translocon protein.

In another operation 120, the process 100 may comprise using at least a subset of the plurality of cell-free precursors from the first portion to form the biological molecule. The cell-free precursors may not comprise the biological molecule. For example, the cell-free precursors may comprise components of the biological molecule but not the completed biological molecule. The cell free precursors may comprise at least a portion of a homogenized cell. For example, the cell-free precursors can be a homogenized lysate of a cell. In another example, the cell-free precursors can be a minimum set of purified recombinant proteins. The cell-free precursors may comprise cellular bodies (e.g., organelles). The cell-free precursors may comprise substrates (e.g., peptides, nucleic acids, sugars, etc.). The cell-free precursors may comprise one or more energy sources (e.g., adenosine triphosphate, etc.). The cell-free precursors may comprise one or more nucleic acids (e.g., deoxyribonucleic acid, ribonucleic acid, etc.). The one or more nucleic acids may encode for the biological molecule.

The cell-free precursors may comprise a plurality of different nucleic acid templates. The plurality of nucleic acid templates may be at least about 2, 5, 10, 50, 100, 500, 1,000, 5,000, 10,000, or more nucleic acid templates. The plurality of nucleic acid templates may comprise at most about 10,000, 5,000, 1,000, 500, 100, 50, 10, 5, 3, or less nucleic acid templates. The plurality of different nucleic acid templates may be introduced to the first portion of the chamber at the same time. The plurality of different nucleic acid templates may cause generation of a plurality of different biological molecules. For example, a plurality of different proteins can be formed and translocated through the membrane. In this example, subsequent to the translocation, the proteins can interact with binding moieties that bind target proteins while the other proteins are washed away. In this example, the bound proteins can be eluted and subsequently sequenced or otherwise used. The forming of the biological molecule may comprise use of one or more enzymes (e.g., nucleic acid polymerases for forming a nucleic acid, ribosomes for forming a protein, etc.). For example, an RNA can be fed into a ribosome and translated into a polypeptide using the ribosome. In another example, a DNA molecule can be translated into an RNA molecule using a polymerase.

In another operation 130, the process 100 may comprise, during or subsequent to operation 120, translocating at least a portion of the biological molecule through the pore into the second portion. The translocating may occur substantially simultaneously to the forming of the biological molecule. For example, the biological molecule may be formed and translocated through the pore almost as it is formed. In this example, the biological molecule may change conformation in the second chamber (e.g., a protein biological molecule can fold in the second chamber). The translocating may occur subsequently to the forming of the biological molecule. For example, the biological molecule can be generated in the first portion and subsequently diffuse to the membrane, where it can then traverse to the second portion by being driving by a SecA ATPase. In this example, a concentration of the biological molecule may be built up to increase diffusion into the second portion. In some cases, chaperones may be present in the first portion to maintain the biological molecule in an unfolded state prior to translocation through the membrane. The translocating may occur co-translationally. For example, a plurality of the cell-free precursors can begin generation of the biological molecule, the biological molecule can be moved to a pore in the membrane, and the biological molecule can be translocated directly after formation into the second portion. The translocating may be active translocation (e.g., energy is used to translocate the biological molecule through the pore). For example, the translocation can comprise use of adenosine triphosphate to provide energy for the translocation. In another example, an electric field may be applied to facilitate diffusion of the biological molecule through the pore. The translocating may be passive translocation (e.g., the biological molecule can be translocated in an absence of input energy).

The biological molecule may undergo one or more conformational changes subsequent to translocating through the pore. The biological molecule may undergo one or more folding transformations subsequent to translocating through the pore. For example, a protein biological molecule can be formed in the first portion and folded in the second portion. The conditions within the first and second portions of the chamber may be different. For example, the conditions in the first portion can be optimized for synthesis of the biological molecule, while conditions in the second portion can be optimized for folding or other conformational changes. Examples of conditions include, but are not limited to ionic strength, presence or absence of chaperone molecules, presence or absence of enzymes (e.g., enzymes configured to confer post-translational modifications), or the like, or any combination thereof.

The supported lipid bilayer may comprise one or more signal peptidase proteins. The signal peptidase proteins may be configured to cleave a portion of the biological molecule subsequent to translocation through the pore. For example, a biological molecule can be generated with an N-terminal signal sequence that is cleaved by a signal peptidase. Examples of signal peptidase subunits include, but are not limited to, SPC3P, SPC2P, SPC1P, SEC11, SPC12, SPC18, SPC21, SPC22/23 and SPC25. Examples of signal peptidases include, but are not limited to, LepA and LepB. The inclusion of the signal peptidase proteins in the supported lipid bilayer may permit biological molecule generation schemes in which biological molecules are generated with translocation signal sequences to facilitate translocation across the supported lipid bilayer that are subsequently removed to generate pure and complete biological molecules. The supported lipid bilayer may comprise one or more signal peptide hydrolase proteins. The signal peptide hydrolase proteins may be configured to digest the signal peptide that may remain in the membrane after it is cleaved by the signal peptidase. The inclusion of the signal peptide hydrolase may reduce buildup of signal peptides in the membrane an improve longevity of the membrane.

Subsequently to operation 130, the process 100 may comprise removing an N-terminal translocation signal sequence from the biological molecule. molecule. For example, a signal peptidase can be used to cleave the N-terminal translocation signal sequence from the biological molecule, thus generating the biological molecule. Alternatively, the biological molecule may be generated without an N-terminal translocation signal sequence. The biological molecule may be generated with other additional sequences (e.g., other signaling sequences, secondary domains, etc.). the other additional sequences may be removed from the biological molecule subsequent to the formation of the biological molecule.

In some cases, operation 140 may be performed. In operation 140, the process 100 may comprise removing the biological molecule from the second portion of the chamber. The removing the biological molecule may comprise destruction of the supported lipid bilayer. For example, pressurized gas can be used to force a solution comprising the biological molecule out of the second portion. The removing the biological molecule may comprise a flow of solvent. For example, a flow-cell apparatus can be used to collect the biological molecule from the second portion. The removing may comprise one or more purification operations. Examples of purification operations include, but are not limited to, chromatographic operations (e.g., affinity chromatography, size exclusion chromatography, ion exchange chromatography), extraction operations (e.g., solvent extractions, salt formation reactions, etc.), centrifugation operations (e.g., filter centrifugation, ultracentrifugation, etc.), filtration operations (e.g., paper filtration, tangential flow filtration, ultrafiltration, diafiltration, etc.), lyophilization operations, magnetic separation (e.g., removal of metal nanoparticle tagged reagents/chaperones, etc.) or the like, or any combination thereof. For example, the removing may comprise passing a solution comprising the biological molecule through a filter and a gel chromatography column. The removing may comprise at least about 0, 1, 2, 3, 4, 5, 6, 7, 8, 9, 10, or more purification operations. The removing may comprise at most about 10, 9, 8, 7, 6, 5, 4, 3, 2, 1, or less purification operations. The removing may comprise no purification operations. Subsequent to the removing, reagents separated from the biological molecule may be reused for formation of other biological molecules.

In another aspect, the present disclosure provides a system for generating a biological molecule. The system may comprise a chamber. The chamber may comprise a first portion configured to comprise a plurality of cell-free precursors of the biological molecule. The chamber may comprise a second portion. The chamber may comprise a porous membrane separating the first portion from the second portion. The porous membrane may comprise a lipid bilayer. The lipid bilayer may comprise one or more translocon proteins. The lipid bilayer and the translocon proteins may be as described elsewhere herein. The biological molecule may be a biological molecule as described elsewhere herein. The lipid bilayer may be a supported lipid bilayer.

The porous membrane may comprise a polymer membrane. The polymer membrane may comprise polysulfone, polyethersulfone, polytetrafluoroethylene, polymethylmethacrylate, polyacrylonitrile butadiene styrene, a polyamide, polylactic acid, polybenzimidazole, polycarbonate, polyether sulfone, polyoxymethylene, polyetherether ketone, polyetherimide, polyethylene, polyphenylene oxide, polyphenylene sulfide, polypropylene, polystyrene, polyvinyl chloride, polyvinylidene fluoride, or the like, or any combination thereof The polymer membrane may be hydrophilic. For example, the porous membrane may comprise hydrophilic polysulfone. The polymer membrane may be hydrophobic. The polymer membrane may be functionalized. For example, a polymer membrane can be treated with ozone to generate surface hydroxy groups on the polymer. The polymer membrane may have a molecular weight cutoff of at least about 1, 5, 10, 15, 20, 25, 30, 35, 40, 45, 50, 55, 60, 65, 70, 75, 80, 85, 90, 95, 100, 110, 120, 130, 140, 150, 175, 200, or more kilodaltons. The polymer membrane may have a molecular weight cutoff of at most about 200, 175, 150, 140, 130, 120, 110, 100, 95, 90, 85, 80, 75, 70, 65, 60, 55, 50, 45, 40, 35, 30, 25, 20, 15, 10, 5, 1, or less kilodaltons. The polymer membrane may have a molecular weight cutoff in a range as defined by any two of the proceeding values. For example, the polymer membrane can have a molecular weight cutoff of about 80 to 110 kilodaltons. The porous membrane may comprise a mesoporous material. Examples of mesoporous materials include, but are not limited to, mesoporous metal oxides (e.g., mesoporous alumina, mesoporous titanium oxide, etc.), mesoporous silica, mesoporous salts (e.g., mesoporous magnesium carbonate, etc.), and mesoporous carbon. The porous membrane may be a treated membrane. Examples of treatments include, but are not limited to, applying one or more other materials to the membrane (e.g., metal plating, polymer coating, etc.), functionalizing the membrane (e.g., applying one or more chemical species to the membrane (e.g., carboxylated polymers, surfactants, passivants, etc.)), or the like, or any combination thereof. The porous membrane may comprise other materials which may comprise pores (e.g., be porous), such as, for example a porous glass substrate, a porous dielectric material substrate, a porous metal substrate, a porous fiber-based substrate (e.g., a paper substrate), or the like, or any combination thereof.

The supported lipid bilayer may comprise one or more proteins. The one or more proteins may be configured to translocate the biological molecule across the membrane. For example, the biological molecule can pass through a pore in the membrane formed by a protein. The one or more proteins may comprise one or more translocon proteins. For example, the one or more translocon proteins may comprise SecYEG. The one or more proteins may comprise one or more injectosomes. The one or more proteins may comprise one or more hemolysins (e.g., alpha-hemolysin). The one or more proteins may comprise one or more pore forming toxins. The one or more proteins may be one or more proteins as described elsewhere herein.

The plurality of cell-free precursors may not comprise one or more cells. The plurality of cell-free precursors may be generated by lysis and homogenization of one or more cells. For example, a plurality of E. coli cells can be lysed, and the contents of the cells can be used as cell-free precursors. The one or more cell-free precursors may comprise deoxyribonucleic acid (DNA), ribonucleic acid (RNA), one or more amino acids, one or more cofactors (e.g., magnesium, iron, vitamins, minerals, etc.), ribosomes, synthetases, nucleases, or the like, or any combination thereof. The DNA and/or the RNA may encode for the biological molecule. For example, the DNA can encode for the amino acids of a polypeptide. Alternatively, the first portion may comprise one or more cells. The cells may be configured to generate the biological molecule, and the membrane can be used to separate the biological molecule from the cells. For example, the cells can secret the biological molecule into solution in the first portion. In this example, the solution can comprise either a chaperone configured to maintain the biological molecule in an unfolded state (e.g., SecB) and an active transport body (e.g., SecA ATPase, a molecular motor) to translate the biological molecule across the lipid bilayer or vesicles (e.g., fusogenic vesicles) configured to shuttle the biological molecule to the lipid bilayer, fuse with the lipid bilayer, and thus transport the biological molecule to the second portion. In this example, an injectosome may be used to transport the biological molecule through the lipid bilayer.

The second portion may comprise a different environment from the first portion. The different environment may be a different temperature, solvent system (e.g., polarity, solvent mixture, etc.), ionic strength, presence or absence of other molecules (e.g., cofactors, binding substrates, etc.), presence of absence of chaperone molecules, presence or absence of post-translational modification enzymes, or the like, or any combination thereof. For example, the second portion may be held at a lower ionic strength than the first portion. In this example, electrostatic screening may be lower in the second portion, thus permitting increased interaction between different portions of the biological molecule.

In another aspect, the present disclosure provides a method for generating a cell-free synthesis chamber. The method may comprise providing a chamber comprising a first portion and a second portion. The first portion and the second portion may be separated by a porous membrane. A solution comprising a plurality of proteoliposomes may be applied to the porous membrane. The plurality of proteoliposomes may comprise a lipid bilayer and one or more translocon proteins. The plurality of proteoliposomes may be reacted with the porous membrane. The reacting may comprise dissociation of the plurality of proteoliposomes to form a supported lipid bilayer on the porous membrane. The supported lipid bilayer may comprise the one or more translocon proteins.

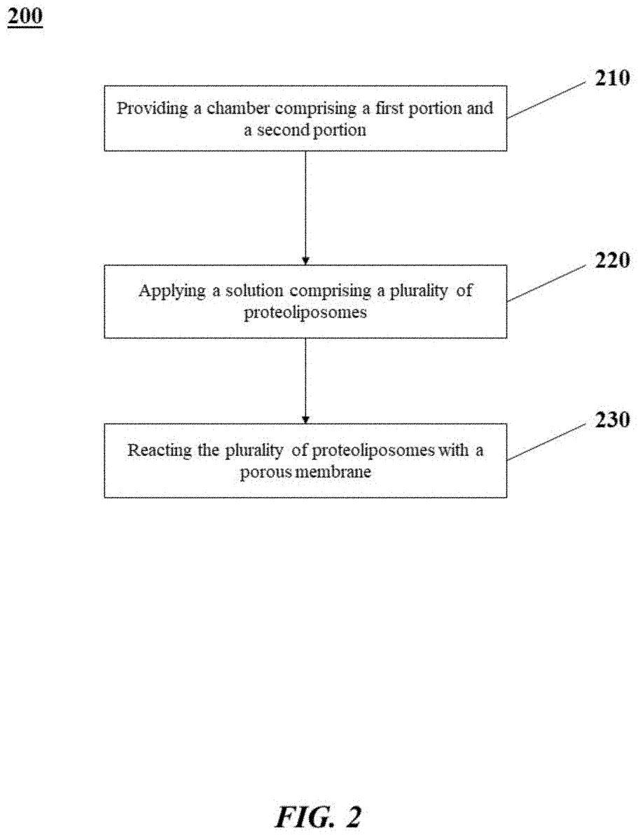

FIG. 2 is an example of a flowchart for a process 200 for generating a cell-free synthesis chamber. In an operation 210, the process 200 may comprise providing a chamber comprising a first portion and a second portion. The first portion may be separated from the second portion by a porous membrane. The chamber may be a chamber as described elsewhere herein. For example, the chamber may be a flow chamber.

In another operation 220, the process 200 may comprise applying a solution comprising a plurality of proteoliposomes. The plurality of the proteoliposomes may comprise a lipid bilayer and one or more translocon proteins. In some cases, the plurality of proteoliposomes may not comprise one or more translocon proteins. For example, the proteoliposomes can be liposomes. In this example, the liposomes can be used to generate a supported lipid bilayer, and subsequent to the forming of the supported lipid bilayer, one or more translocon proteins may be added to the supported lipid bilayer. Other ways of forming supported lipid bilayers may be used as well, such as, for example, lipid stacking followed by plasma etching.

The solution may comprise a plurality of liposomes without the one or more translocon proteins. The liposomes without the one or more translocon proteins may be of a same composition as the plurality of proteoliposomes. For example, the liposomes and the proteoliposomes can both comprise POPC. The concentration of translocon proteins may be tuned to a predetermined value by adjusting the ratio of the liposomes to the proteoliposomes. For example, a lipid bilayer with dilute translocon proteins can be formed by generating a solution with more liposomes than proteoliposomes and applying the solution to the porous membrane.

The proteoliposomes and/or the liposomes may be substantially homogeneous in size. The proteoliposomes and/or the liposomes may have a size distribution of at least about 1%, 2%, 3%, 4%, 5%, 6%, 7%, 8%, 9%, 10%, or more. The proteoliposomes and/or the liposomes may have a size distribution of at most about 10%, 9%, 8%, 7%, 6%, 5%, 4%, 3%, 2%, 1%, or less. The proteoliposomes and/or the liposomes may be generated by extrusion through a pore. The proteoliposomes and/or the liposomes may be generated by rehydration of lipids in an aqueous solution. The proteoliposomes and/or the liposomes may be generated by sonication. The proteoliposomes and/or the liposomes may be formed by other processes such as, for example, those described in “Novel methods for liposome preparation” by Patil et al., Chemistry and Physics of Lipids Volume 177, January 2014, Pages 8-18 DOI number 10.1016/j.chemphyslip.2013.10.011, which is incorporated by reference in its entirety. The proteoliposomes and/or the liposomes may have a size of at least about 10, 50, 100, 250, 500, 1,000, or more nanometers. The proteoliposomes and/or the liposomes may have a size of at most about 1,000, 500, 250, 100, 50, 10, or less nanometers.

The proteoliposomes may be generated by incubation of liposomes with cell-free precursors of the translocon proteins. For example, the translocon proteins can be generated in a cell-free reaction using RNA and/or DNA that encodes for the translocon proteins and cell-free ribosomes and the liposomes can be introduced into the cell-free reaction mixture and incubated until the translocon proteins are incorporated into the liposomes. The proteoliposomes may be generated by rehydrating dried translocon proteins with a solution comprising liposomes. For example, a solution comprising translocon proteins can be lyophilized and subsequently rehydrated in a solution comprising proteoliposomes. The proteoliposomes may be generated by a detergent exchange method. For example, proteins solubilized in a detergent can be added to a solution comprising micelles, and the proteins can be exchanged from the detergent to incorporate into the micelles. In another example, proteins solubilized in a detergent can be added to a solution comprising unilamellar vesicles, and the proteins can be exchanged from the detergent to incorporate into the unilamellar vesicles. The proteoliposomes may be generated by mixing a translocon protein solution with a liposome solution. For example, the translocons can integrate into the liposomes in solution.

In another operation 230, the process 200 may comprise reacting the plurality of proteoliposomes with the porous membrane. The reacting may comprise dissociation of the plurality of proteoliposomes to form a supported lipid bilayer on the porous membrane. The supported lipid bilayer may comprise the one or more translocon proteins. In some cases, the supported lipid bilayer may be generated on the porous membrane separate from a chamber. For example, the supported lipid bilayer may be generated on a porous membrane in a reaction vessel configured for formation of supported lipid bilayers. In this example, the porous membrane comprising the supported lipid bilayer can be removed from the reaction vessel and placed within a chamber as described elsewhere herein. In some cases, the supported lipid bilayer may be formed without any translocon proteins. The translocon proteins may be added to the supported lipid bilayer subsequent to the formation of the supported lipid bilayer. For example, a supported lipid bilayer can be formed on the porous membrane and a solution comprising the translocon proteins can be introduced to the supported lipid bilayer and the translocon proteins can integrate into the supported lipid bilayer. The supported lipid bilayer may be generated from inverted membrane vesicles. The inverted membrane vesicles may be derived from one or more cells. For example, E. coli can be configured to generate translocon proteins, either natively or with genetic engineering, and the cells of the E. coli can be transformed into inverted membrane vesicles that may subsequently be reacted to form a supported lipid bilayer comprising translocon proteins. The process of generating a supported lipid bilayer from inverted membrane vesicles may be similar to generating a supported lipid bilayer from proteoliposomes. For example, the inverted membrane vesicles may be reacted with a porous substrate to form a supported lipid bilayer on the porous substrate. The membrane may be a lipid bilayer. The lipid bilayer may be supported by one or more substrates. In some examples, the lipid bilayer is supported by substrates (e.g., sandwiched between two substrates). The membrane may be a solid-state membrane, such as, for example, a dielectric. The solid-state membrane may be formed of a silicon oxide or a silicon nitride, for example.

In another aspect, the present disclosure provides a method for generating a polypeptide. The method may comprise using a cell-free solution comprising a deoxyribonucleic acid molecule encoding the polypeptide to generate a ribonucleic acid molecule. The ribonucleic acid molecule may be used to generate the polypeptide. The polypeptide may be directed through a pore disposed in a membrane.

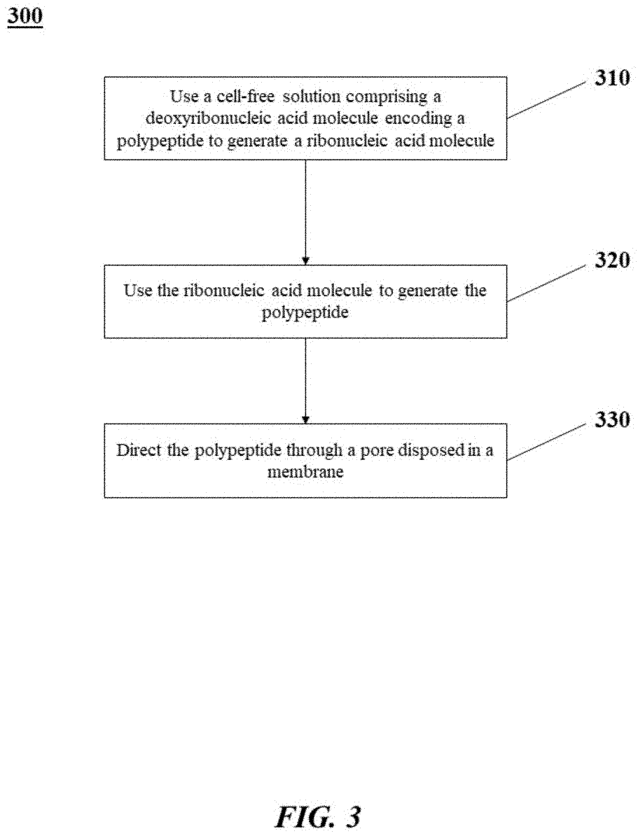

FIG. 3 is an example flowchart for a process 300 for generating a polypeptide. In an operation 310, the process 300 may comprise using a cell-free solution comprising a deoxyribonucleic acid molecule encoding a polypeptide to generate a ribonucleic acid molecule. Alternatively, the ribonucleic acid molecule may be introduced to the cell-free solution already generated. For example, a ribonucleic acid encoding a protein can be introduced to a cell-free solution that does not comprise DNA.

In another operation 320, the process 300 may comprise using the ribonucleic acid molecule to generate the polypeptide. The generation may comprise use of one or more cellular bodies (e.g., ribosomes, peptidases, etc.).

In another operation 330, the process 300 may comprise directing the polypeptide through a pore disposed in a membrane. The pore may comprise a protein. The protein may comprise a translocon protein. The membrane may be a supported lipid bilayer. The membrane may be another membrane as described elsewhere herein. The pore and the membrane may be as described elsewhere herein. The polypeptide may comprise a non-native N-terminal signal sequence. For example, the RNA may encode for a non-wildtype polypeptide that has been configured to comprise a terminal signal sequence. The terminal signal sequence may be configured to be removed by a signal peptidase protein.

The membrane may comprise a supported lipid bilayer. The membrane may not be a part of a micelle. For example, the membrane may not be a membrane free in solution. The membrane may be planar. For example, the membrane may be a planar supported lipid bilayer on a support. The membrane may be substantially planer. For example, the membrane can be applied to a rough support. The pore may comprise one or more translocon proteins. The one or more translocon proteins may be as described elsewhere herein. The membrane may comprise one or more signal peptidase proteins and/or one or more other proteins as described elsewhere herein. The membrane may be rolled into a hollow fiber configuration (e.g., rolled into a tube).

Subsequent to operation 330, the polypeptide may be present at a purity of at least about 20%, 30%, 40%, 50%, 55%, 60%, 65%, 70%, 75%, 80%, 85%, 90%, 95%, 96%, 97%, 98%, 99%, or more. Subsequent to operation 330, the polypeptide may be present at a purity of at most about 99%, 98%, 97%, 96%, 95%, 90%, 85%, 80%, 75%, 70%, 65%, 60%, 55%, 50%, 40%, 30%, 20%, or less. The polypeptide may be present at one of the aforementioned purities without additional purification operations. For example, subsequent to moving through the pore, the polypeptide can be at a purity of at least about 60%. The polypeptide may be used without further purification subsequent to operation 330. The purity may be a purity of the molecular weight of the biological molecule (e.g., a size distribution of the completed biological molecule), a molarity of the biological molecule, a ratio of the biological molecule to other molecules in solution, a ratio of the biological molecule to other biological molecules in solution, or the like, or any combination thereof. The purity may be a purity in a second portion of a chamber as described elsewhere herein. The purity may be a purity of biological molecules in a membrane as described elsewhere herein.

Operations 310-330 may be performed within a time period of at least about 30 seconds, 1 minute (m), 5 m, 10 m, 15 m, 30 m, 1 hour (h), 2 h, 3 h, 4 h, 5 h, 6 h, 7 h, 8 h, 9 h, 10 h, 12 h, 18 h, 24 h, 48 h, 72 h, 96 h, or more. Operations 310-330 may be performed within a time period of at most about 96 h, 72 h, 48 h, 24 h, 18 h, 12 h, 10 h, 9 h, 8 h, 7 h, 6 h, 5 h, 4 h, 3 h, 2 h, 1 h, 30 m, 15 m, 10 m, 5 m, 1 m, 30 s, or less.

In another aspect, the present disclosure provides a system for generating a biological molecule. The system may comprise a chamber. The chamber may comprise a first portion configured to contain a plurality of cell-free precursors of the biological molecule. The chamber may comprise a second portion. The chamber may comprise a porous membrane separating the first portion from the second portion. The porous membrane may comprise a supported lipid bilayer. The supported lipid bilayer may comprise one or more signal peptidase proteins.

The one or more signal peptidase proteins may comprise one or more signal peptidase proteins as described elsewhere herein. For example, the one or more signal peptidase proteins may comprise LepB. The supported lipid bilayer may comprise one or more translocon proteins as described elsewhere herein. For example, the supported lipid bilayer may be configured to permit translocation of the biological molecule through the porous membrane through the one or more translocon proteins.

FIGS. 4A-4D are examples of a method for generating a flow cell chamber. A flow chamber 601 may comprise two or more flow ports 602. The chamber 601 may comprise at least about 2, 3, 4, 5, 6, 7, 8, 9, 10, or more flow ports. The chamber 601 may comprise at most about 10, 9, 8, 7, 6, 5, 4, 3, or fewer flow ports. For example, the chamber can comprise two flow ports on one side of a membrane and two flow ports on the other side of the membrane. The flow ports may be in fluidic communication with one or more reservoirs (e.g., reagent reservoirs, wash reservoirs, etc.), waste handling (e.g., waste disposal), characterization instrumentation (e.g., chromatography, mass spectrometry, nuclear magnetic resonance, optical, etc.), lab-on-a-chip functionalities (e.g., those described elsewhere herein), other chambers configured to generate other biomolecules (e.g., the output of a first chamber is a portion of the cell-free reaction mixture of the second chamber), or the like, or any combination thereof. The flow ports may be on a same side of the chamber. For example, all of the flow ports can be on the side of the chamber in order to permit easy insertion and removal from a larger system.

The chamber may comprise a first portion 604 and a second portion 605 separated by a membrane 603. The membrane may be a membrane as described elsewhere herein. For example, the membrane may comprise a mesoporous membrane. In the process of generating a supported lipid bilayer on the membrane, a plurality of translocon-containing proteoliposomes can be flowed into the first and/or second portions of the chamber via the flow ports 602. In the example of FIG. 4B, the solution 606 can be flowed into the first portion. The solution may be described as elsewhere herein. For example, the solution may comprise a plurality of both proteoliposomes and liposomes. The solution may be reacted with the membrane as described elsewhere herein. For example, the solution can be incubated with the membrane and reacted to form a supported lipid bilayer on the membrane. The supported lipid bilayer may comprise one or more translocon and or signal peptidase proteins as described elsewhere herein.

After the reaction to form a supported lipid bilayer 607 comprising the translocon and/or signal peptidase proteins, a cell-free reaction mixture 608 may be introduced into the chamber via one or more flow ports as shown in FIG. 4C. The cell-free reaction mixture may be flowed into the first or the second portion. The cell-free reaction mixture may be as described elsewhere herein. The chamber may be configured to hold the cell-free reaction mixture under conditions sufficient for the formation of one or more biological molecules 609 as described elsewhere herein. The one or more biological molecules may translocate through the membrane to the other portion of the chamber. Once in the other portion, the biological molecules may remain in the other portion when not subjected to a flow. Alternatively, if a flow is present in the other portion, the biological molecules may be flowed out of the chamber through one of the flow ports.

FIG. 4D is an example of a wash operation subsequent to the formation of the one or more biological molecules. A wash 610 may be flowed into the chamber to wash the cell-free reaction mixture and/or the biological molecules out of the chamber. The was operation may comprise flow of fluid to one portion of the chamber but not the other portion of the chamber. For example, a pressurized wash can be applied to the first portion, and the biological molecule can be driven to the second portion by the pressure. In another example, the second portion can be washed to remove the biological molecule while the first portion is not washed. Subsequent to the wash, the chamber may be reused for the generation of the same or different biological molecules. For example, the chamber can be treated with a DNase and/or an RNase to remove remaining reactants. In this example, a DNase and/or RNase inhibitor can the be introduced prior to reintroduction of the cell-free precursors.

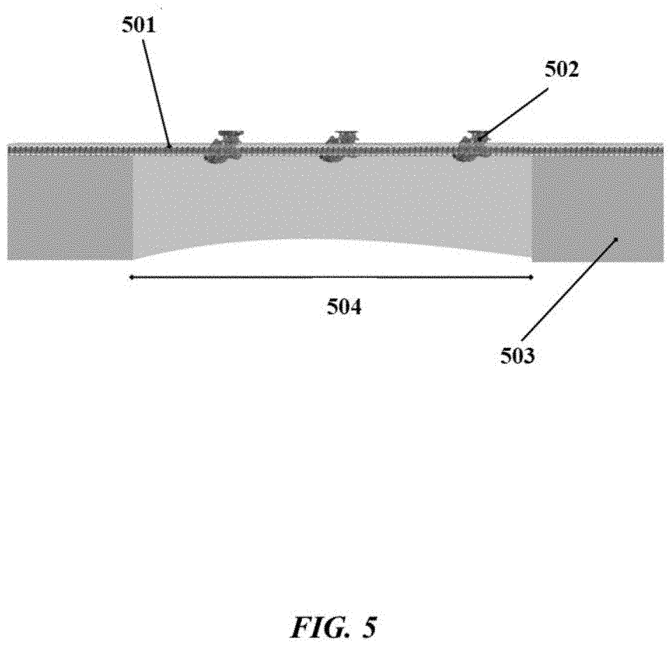

FIG. 5 is an example of a supported lipid bilayer 501 comprising proteins 502. Although the supported lipid bilayer 501, as illustrated, comprises three proteins 502, the supported lipid bilayer 501 may comprise at least 1, 2, 3, 4, 5, 6, 7, 8, 9, 10, 20, 30, 40, 50, 100, 200, 300, 400, 500, 1000 or more proteins 502. The supported lipid bilayer may be a supported lipid bilayer as described elsewhere herein. For example, the supported lipid bilayer may comprise 1-palmitoyl-2-oleoyl-sn-glycero-3-phosphocholine. The proteins may comprise translocon proteins as described elsewhere herein, signal peptidase proteins as described elsewhere herein, or a combination thereof. The positioning of the proteins above the pore may permit the transit of biological molecules through the bilayer 501 through pores in the translocon proteins.

The supported lipid bilayer may be supported on membrane 503. The membrane may be a membrane as described elsewhere herein. For example, the membrane can comprise mesoporous alumina, mesoporous silica, or mesoporous polysulfone. The membrane may comprise one or more pores 504. The pore may have a size of at least about 10 nanometers (nm), 25 nm, 50 nm, 75 nm, 100 nm, 150 nm, 200 nm, 250 nm, 500 nm, 750 nm, 1,000 nm, or more. The pore may have a size of at most about 1,000 nm, 750 nm, 500 nm, 250 nm, 200 nm, 150 nm, 100 nm, 75 nm, 50 nm, 25 nm, 10 nm, or less.

The membrane 503 may comprise additional structures such as, for example, electrodes, electrical leads, temperature sensors, proteins, cellular bodies, organelles, or the like, or any combination thereof. For example, protein generating organelles can be tethered to the membrane adjacent to the pores to permit translocation of the protein upon generation by the organelle. In another example, the membrane can comprise electrodes configured to generate an electric field to direct flow of biological molecules through the pore.

### Computer Systems

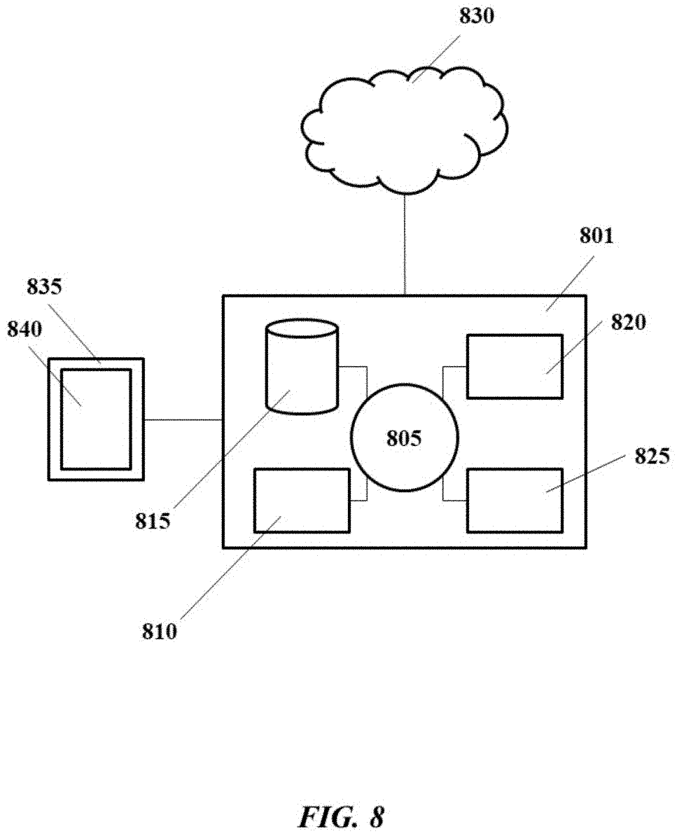

The present disclosure provides computer systems that are programmed to implement methods of the disclosure. FIG. 8 shows a computer system 801 that is programmed or otherwise configured to perform methods and regulate systems of the present disclosure. The computer system 801 can regulate various aspects of the present disclosure, such as, for example, methods of generating biological molecules or generating cell-free synthesis chambers. For example, a computer system can be configured to control the conditions for the formation of a biological molecule within the chamber. In another example, a computer system can regulate the conditions of the forming of a cell-free synthesis chamber. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries, and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a control panel for inputting predetermined properties of a biological molecule. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, cycle a cell-free reaction chamber to generate a biological molecule.

### EXAMPLES

The following examples are illustrative of certain systems and methods described herein and are not intended to be limiting.

### Example 1

### Preparation of a Chamber Comprising a Supported Lipid Bilayer

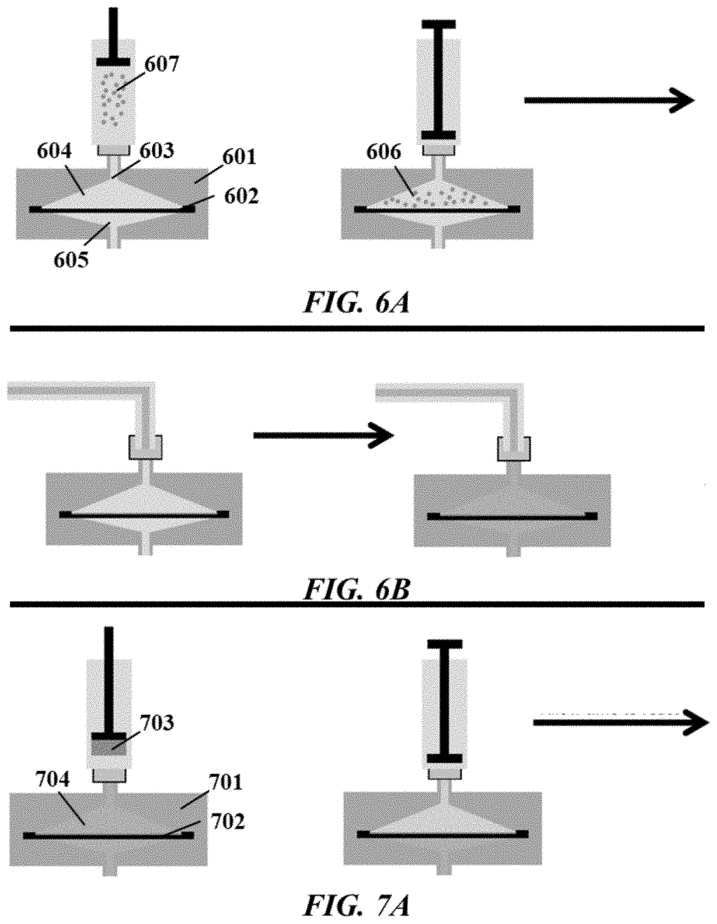

FIGS. 6A and 6B are examples of a process for generating a chamber 601 comprising a membrane 602 and using the chamber to generate a biological molecule. The chamber may comprise an inlet 603. The inlet may be configured to connect to a vessel 607 (e.g., a syringe, a tube, etc.). The vessel may be configured to introduce a plurality of proteoliposomes to the first portion 604 of the chamber 601.

The proteoliposomes may be formed by evaporation of chloroform solvent from POPC lipids using nitrogen gas flow followed by application of vacuum. The dry POPC can be rehydrated in an aqueous buffer and extruded through 100 nm pores to generate liposomes. The liposomes can then be incubated with a cell-free transcription/translation solution with DNA encoding for SecY, SecE, and SecG for 3 hours at about 37 degrees Celsius to form SecYEG impregnated proteoliposomes.

In this example, a 25-millimeter disc of hydrophilic polysulfone can be used as the membrane 602. The membrane can be soaked in a 50% ethanol solution to expand the polymer and subsequently washed in water to remove the ethanol. The membrane 602 can then be placed into the chamber 601, and a solution 606 comprising SecYEG proteoliposomes as well as additional liposomes can be deposited into the first portion 604 via the vessel 616. The solution can be incubated for 3 hours at ambient temperature to form a SecYEG impregnated supported lipid bilayer on the membrane 602. The solution 606 may be a buffer solution (e.g., pH buffered, ionic strength buffered, etc.).

After formation of the supported lipid bilayer, a flux test may be performed using a pressurized water line 607. The pressurized water line may be at a pressure of 1 bar, and the flow of water across the membrane 602 may be measured and recorded in order to determine an extent of lipid bilayer coverage of the membrane. Other examples of quality control tests include, but are not limited to fluorescence microscopy and atomic force microscopy. For example, a fluorescence microscopy image can be used to confirm the presence of the lipids of the lipid bilayer. In this example, photobleaching can be used to confirm that the lipids are a bilayer instead of immobilized unruptured liposomes. If the degree of coverage is determined to be acceptable, the chamber and membrane may be used in the formation of biological molecules.

### Example 2

### Preparation of a Biological Molecule

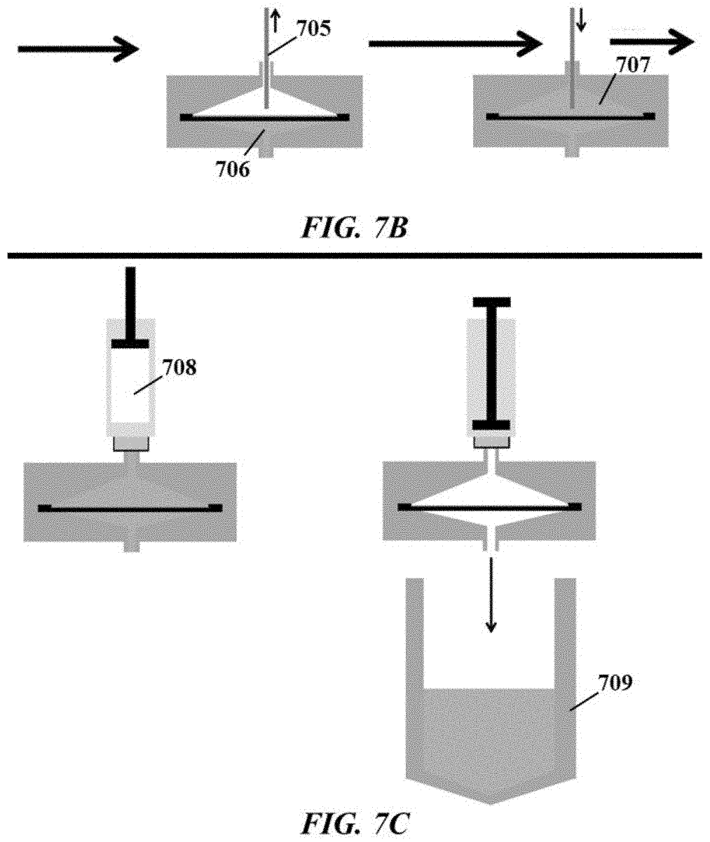

FIGS. 7A-7C are examples of a process for generating a biological molecule. Into a first portion 704 of a chamber 701 comprising a membrane 702, such as the chamber generated in Example 1, a cell-free reaction mixture 703 can be injected. The cell free reaction mixture may be generated by homogenizing E. coli cells. The lysate may be fractionated using a plurality of 12,000 rcf centrifugations to produce the cell-free reaction mixture. The mixture may be centrifuged again at 135,000 rcf to remove inverted membrane vesicles as well and can be stored at −80° C. for future use.

When the lysate 703 is added to the chamber 701, one or more nucleic acid sequences encoding for the biological molecule may be added as well. For example, DNA encoding for beta galactosidase, TrxA or OmpA can be added to form those proteins, though other proteins may be formed by similar methods. The one or more nucleic acid sequences may comprise a portion encoding for an N-term translocation signal sequence. To the lysate, additional components such as energy molecules (e.g., adenosinetriphosphate) and substrates (e.g., peptides) can be added. The chamber can be held at 37° C. to allow for the generation of the product biological molecule and permit the biological molecule to translocate through the membrane 702.

Subsequently to the formation and translocation of the biological molecule, the cell-free solution may be removed from the first portion 704 via a pipette or other fluid transport apparatus 705. At this point, the biological molecule can reside in the second portion 706 of the chamber 701. The first portion 704 may be rinsed one or more times to remove any additional cell-free precursors and leave a clean solution 707 in the first portion. The rise may be at a low flow rate to avoid shearing the supported lipid bilayer.

To recover the biological molecule, pressurized gas 708 (e.g., air, nitrogen, etc.) may be flushed into the chamber and rupture the supported lipid bilayer. The contents of the first and second portions may then be collected into a vessel 709 and removed. Alternatively, a fluid transport pipe can be used in place of the vessel to remove the biological molecule from the chamber.

Though described herein with respect to a single inlet and outlet chamber, the methods of the examples can be utilized in a flow cell setup. An example of a flow cell setup can be found in FIGS. 4A-4D. In a flow cell setup, both cell-free solutions as well as product biological molecules can be constantly flowed through the flow cell. An advantage of a flow cell setup is that continuous production of biological molecules can be achieved. Additionally, a flow cell setup can have improved speed of processing as well as reduced machinery costs.

### Example 3

### Automated Testing of Protein Synthesis

A computer system operatively coupled to the systems described elsewhere herein can be used to provide an automated design and testing platform for biomolecule synthesis. Though described herein with respect to protein synthesis, other biological molecules as described elsewhere herein may be formed as well.

Due to the relatively short processing times of methods and systems described elsewhere herein (e.g., about 3 hours, about 5 minutes, about 30 seconds, etc.), a continuous flow system can be generated with, for example, 100 chambers each configured to produce about 0.01 mg of protein per hour for a total system rate of 1 mg per hour. The computer can determine based on analytical instruments coupled to the chambers, if each chamber of the system can (i) continue expressing the protein product of that chamber to generate additional protein for analysis or (ii) start making a different protein (e.g., a different protein entirely or a protein generated by at least one other chamber). The decision to stop (ii) may be based on having already collected enough information on the protein to know the value of continuing production of that protein. The decision in increase the number of chambers generating a particular protein can be made by determining if the protein is promising as determined by analysis performed on the chambers currently forming the protein. By directing additional chambers to form the protein, the protein can be supplied in higher quantities and/or faster.

In another example, a chamber can be configured to continually generate a protein, and the results of that synthesis can be monitored by a computer operatively coupled to the chamber. The reaction conditions of the chamber can be changed, and the effect of those changes can be tracked by the computer. In this way, the synthesis that the chamber is undertaking can be optimized in real time to produce an increase in the efficiency of that synthesis process.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A method for generating a biological molecule, comprising:
providing a chamber comprising a first portion comprising containing a plurality of cell-free precursors of said biological molecule, a second portion, and a membrane disposed between said first portion and said second portion, wherein said membrane
allows said biological molecule to be formed from at least a subset of said plurality of cell-free precursors, and
to be translocated through and released from the membrane into said second portion.

2. The method of claim 1, wherein said membrane comprises a lipid bilayer.

3. The method of claim 2, wherein said membrane comprises a support to support said lipid bilayer is a supported lipid bilayer thereon.

4. The method of claim 2, wherein said lipid bilayer comprises one or more translocon proteins.

5. (canceled)

6. (canceled)

7. The method of claim 1, wherein said removing does not comprise a purification operation.

8. The method of claim 1, wherein said biological molecule further comprises an N-terminal translocation signal sequence.

9. The method of claim 8, wherein, after said biological molecule is translocated and released into the second portion, said N-terminal translocation signal sequence is removed from said biological molecule.

10. The method of claim 1, wherein said translocating occurs substantially simultaneously to said forming said biological molecule.

11. The method of claim 1, wherein said translocating occurs subsequently to said forming said biological molecule.

12. The method of claim 1, wherein said translocating occurs co-translationally.

13. The method of claim 1, wherein said biological molecule is a polypeptide.

14. The method of claim 13, wherein said polypeptide is a protein, and wherein at least a portion of said protein is folded in said second portion.

15. The method of claim 1, wherein said membrane comprises a pore, and said pore has a cross section that is larger than a cross section of said biological molecule.

16. The method of claim 1, wherein said chamber is a part of a flow channel

17. The method of claim 1, wherein said cell-free precursors do not comprise said biological molecule.

18. The method of claim 1, wherein said translocating of said biological molecule comprises translocating of an entirety of said biological molecule through sad pore and into said second portion.

19. The method of claim 1, wherein said translocating of said biological molecule occurs during said forming of said biological molecule.

20. The method of claim 1, wherein said translocating of said biological molecule occurs subsequent to said forming of said biological molecule.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. A system for generating a biological molecule, comprising:
a chamber comprising
a first portion configured to comprise a plurality of cell-free precursors of said biological molecule;
a second portion; and
a porous membrane separating said first portion from said second portion, wherein said porous membrane comprises a lipid bilayer, and wherein said lipid bilayer comprises one or more signal peptidase proteins.

48. The system of claim 47, wherein said one or more signal peptidase proteins comprise LepB.

49. (canceled)

50. (canceled)

51. (canceled)

52. (canceled)

53. (canceled)

54. (canceled)

55. (canceled)

56. (canceled)
